(19) United States
(12) Patent Application Publication
(10) Pub. No.: US 2012/0329876 A1
(43) Pub. Date: Dec. 27, 2012

(54) METHOD AND COMPOSITION FOR STABLE AND CONTROLLED DELIVERY OF (-)-HYDROXYCITRIC ACID

(75) Inventors: Dallas L. CLOUATRE, Santa Monica, CA (US); James M. Dunn, (US); Caroline Dunn, Littleton, CO (US)

(73) Assignee: Glykon Technologies Group, LLC

(21) Appl. No.: 13/605,616

(22) Filed: Sep. 6, 2012

Related U.S. Application Data
(63) Continuation of application No. 13/353,961, filed on Jan. 19, 2012.
Application No. 13/353,961 is a continuation of application No. 12/627,579, filed on Nov. 30, 2009, now abandoned.
Application No. 12/627,579 is a continuation of application No. 11/287,905, filed on Nov. 28, 2005, now abandoned.
Application No. 11/287,905 is a continuation of application No. PCT/US2004/017187, filed on May 28, 2004.
Application No. PCT/US2004/017187 is a continuation-in-part of application No. 10/447,992, filed on May 29, 2003, now abandoned.

(51) Int. Cl. A61K 31/194; A61P 3/00

(52) U.S. Cl. 514/574

(57) ABSTRACT

The present invention provides stable encapsulated (−)-hydroxycitric acid (“HCA”)-containing compositions and methods of making the same. A method is provided by which the hygroscopic salts of HCA in their relatively pure and active forms, including especially the potassium salt, but also including the sodium salt, are rendered non-hygroscopic and stable (that is, not prone to lactonization, not readily subject to attachment to ligands which inhibit absorption or lead to excretion, and so forth) such that these HCA salts might be included in dry delivery formats, liquid delivery and in controlled-release vehicles. The nonhygroscopic salts of HCA and its derivatives likewise may be protected against acid degradation, lactonization and undesirable ligand binding when exposed to acidic environments or other challenging conditions. The method taught herein can be employed to reduce the polar/ionic qualities of HCA salts and derivatives when presented to the intestinal lumen to provide advantages in absorption.

### CROSS-REFERENCE TO RELATED APPLICATIONS

This application is a continuation-in-part of U.S. Ser. No. 10/447,992, filed May 29, 2003, the contents of which are hereby incorporated herein by reference in its entirety.

### FIELD OF THE INVENTION

The present invention relates to stable microencapsulated/coated (−)-hydroxycitric acid compositions and methods of making the same.

### BACKGROUND OF THE INVENTION

(−)-Hydroxycitric acid (HCA) is a naturally-occurring acid found in the fruit of members of the plant genus Garcinia. HCA can affect the metabolic functions of mammals, including humans. HCA, as well as several synthetic derivatives of citric acid, can inhibit the production of fatty acids from carbohydrates, suppress appetite, and inhibit weight gain (Sullivan et al., American Journal of Clinical Nutrition 1977; 30: 767). Numerous other benefits have been attributed to the use of HCA, including, but not limited to, an increase in the metabolism of fat stores for energy and an increase in thermogenesis (the metabolism of energy sources to produce body heat in an otherwise wasteful cycle).

Free HCA, calcium, magnesium and potassium salts of HCA (i.e., hydroxycitrates, also referred to as HCA) and poorly characterized mixtures of two or more of these minerals were sold in the American market. Calcium HCA and sodium HCA salts have been sold as early as 1994. Most of the commercial preparations of HCA sold to date consist of calcium salts of varying degrees of purity or, more recently, poorly characterized mixtures of calcium HCA and potassium HCA salts.

Therapeutic use of HCA salts has been limited, however, by their poor absorption and chemical instability at acidic pH, e.g., inactivation of HCA salts via lactonization upon exposure to the acidic milieu of the mammalian gut. HCA is extremely hygroscopic, in both its preferred form as potassium HCA salt and in its secondarily preferred form as sodium HCA salt. As such, HCA in its more biologically active forms can be only be maintained as a powder under controlled conditions.

Prior methods to manipulate HCA salts failed to accommodate its instability in acid and hygroscopic nature. Without special precautions, HCA, in its free acid form and in its potassium and sodium salt forms, will bind to numerous other compounds. The binding of HCA to other compounds can affect its bioavailability to a subject, e.g., as a result HCA is less assimilated by a subject.

Prior methods to formulate the preferred salts of HCA (i.e., potassium HCA and sodium HCA) have been limited because they did not yield a formulation of HCA that was fully stable and workable as capsules, tablets, powders, in beverages or prepared snacks, or in controlled release vehicles. Accordingly, there remains a need for HCA-containing compounds suitable for inclusion in dry delivery formats, liquid delivery and in controlled-release vehicles.

### BRIEF SUMMARY OF THE INVENTION

The present invention provide stable, non-hygroscopic HCA-containing compounds (e.g., potassium HCA) useful for tableting, microencapsulation, the production of controlled-release vehicles and incorporation into dry powders. In one embodiment of the invention, the HCA-containing compound is formulated in a dry delivery system. The dry delivery systems include, e.g., a tablet; dry powder; and dry meal replacement mixture. In another embodiment of the invention, the HCA-containing compound is formulated in a liquid delivery system. The liquid delivery systems include (e.g., a capsule); caplet; and beverage. In yet another embodiment of the invention, the HCA-containing compound is formulated in a controlled-release system. The controlled-release system includes, e.g., a tablet; caplet; and capsule.

In one embodiment of the invention, the HCA-containing compounds of the invention include HCA, one or more absorption-enhancer/controlled-release agents and one or more rate-controlling excipients. The HCA can include, e.g., HCA free acid; HCA salts; HCA derivatives; or any combination thereof. In one embodiment of the invention, the HCA is present from about 1.0% to about 80% of the total weight of the HCA-containing compound. In one embodiment of the invention, the HCA is present from about 5% to about 70% of the total weight of the HCA-containing compound. In one embodiment of the invention, the HCA is present from about 10% to about 60% of the total weight of the HCA-containing compound.

The absorption-enhancer/controlled-release agents can include, e.g., d-alpha-tocopheryl polyethylene glycol succinate (TPGS); Lubritab®; volcanic oils; high viscosity grades of conjugated polyethylene glycol; ethylcellulose, carboxymethylcellulose, cellulose propionate; cellulose acetate propionate; cellulose acetate butyrate; cellulose acetate phthalate (CAP); cellulose triacetate; hydroxypropyl-methylcellulose phthalate; polymethyl methacrylate; polyethyl methacrylate; polybutyl methacrylate; polyisobutyl methacrylate; polyhexyl methacrylate; polylsodecyl methacrylate; polylauryl methacrylate; polyphenyl methacrylate; polymethyl acrylate; polyisopropyl acrylate; polyisobutyl acrylate; polyoctadecyl acrylate; polyethylene; polyethylene low density; polyethylene high density; polypropylene; polyethylene oxide; polyethylene terephthalate; polyvinyl isobutyl ether; polyvinyl acetate; polyvinyl acetate phthalate; polyvinyl chloride; polyurethane; other copolymers of acrylic and methacrylic and esters; waxes; shellac; zein; hydrogenated vegetable oils; polyvinyl alcohol; polyvinylpyrrolidone; methyl cellulose; hydroxypropyl cellulose; hydroxpropylmethyl cellulose or polyethylene glycol; or a mixture thereof. In one embodiment of the invention, the one or more absorption-enhancer/controlled-release agents are present from about 1.0% to about 50% of the total weight of the HCA-containing compound. In one embodiment of the invention, the one or more absorption-enhancer/controlled-release agents are present from about 1.0% to about 40% of the total weight of the HCA-containing compound. In one embodiment of the invention, the one or more absorption-enhancer/controlled-release agents are present from about 1.0% to about 30% of the total weight of the HCA-containing compound.

The rate-controlling excipients can include, e.g., Eastacryl; Kollicoat® IR (polyvinylalcohol-polyethyleneglycol graft-copolymer); cellulose acetate phthalate; Kollicoat® SR; ethyl cellulose; Eudragit® (family of acrylate and methacrylate-based coatings); zein (vegetable protein); acrylic polymers; polyvinyl acetate phthalate; hydroxymethylpropylmethyl cellulose phthalate; cellulose acetate trimalleate; acrylic polymer plasticizers; polymers of polylactic acid; polymers of glycolic acid, and mixtures thereof; Primogel; Pruv™ (stearyl fumarate sodium); citrate esters; triethyl citrate; propylene glycol; and dibutyl sebacate. In one embodiment of the invention, the one or more rate-controlling excipients are present from about 0.0001% to about 60% of the total weight of the HCA-containing compound. In one embodiment of the invention, the one or more rate-controlling excipients are present from about 0.001% to about 50% of the total weight of the HCA-containing compound. In one embodiment of the invention, the one or more rate-controlling excipients are present from about 0.01% to about 25% of the total weight of the HCA-containing compound.

In one embodiment of the invention, the chloride concentration of the HCA-containing compound is less than about 2.5% of the total weight of the HCA-containing compound. In one embodiment of the invention the chloride concentration of the HCA-containing compound is less than about 1.0% of the total weight of the HCA-containing compound. In one embodiment of the invention, the chloride concentration of the HCA-containing compound is less than about 0.5% of the total weight of the HCA-containing compound. In one embodiment of the invention, the total halogen content as chloride of the HCA-containing compound is less than about 2.9% of the total weight of the HCA-containing compound. In one embodiment of the invention, the total halogen content as chloride of the HCA-containing compound is less than about 1.0% of the total weight of the HCA-containing compound. In one embodiment of the invention, the total halogen content as chloride of the HCA-containing compound is less than about 0.6% of the total weight of the HCA-containing compound.

In one aspect of the invention, the HCA-containing compound include HCA, one or more absorption-enhancer/controlled-release agents, one or more rate-controlling excipients, and one or more lubricants. The lubricants include, e.g., magnesium stearate, calcium stearate; sodium stearate, glycerol monostearate; stearic acid; Lubritab®; hydrogenated vegetable oils; waxes; talc; boric acid; sodium benzoate; sodium acetate; sodium chloride; DL-leucine; sodium oleate; sodium lauryl sulfate; magnesium lauryl sulfate and polyethylene glycols and kaolin. In one embodiment of the invention, the one or more lubricants the are present from about 0.0001% to about 10% of the total weight of the of the HCA-containing compound. In one embodiment of the invention, the one or more lubricants are present from about 0.001% to about 10% of the total weight of the of the HCA-containing compound. In one embodiment of the invention, the one or more lubricants are present from about 0.01% to about 5% of the total weight of the of the HCA-containing compound.

In one aspect of the invention, the HCA-containing compound include HCA, one or more absorption-enhancer/controlled-release agents, one or more rate-controlling excipients, and one or more bulking agents/binders. The bulking agents/binders include, e.g., starch paste; acacia; sucrose; poly vinyl pyrrolidone (PVP); hydroxy proplyl methyl cellulose (HPMC); methyl cellulose; gelatin; potato starch; micro crystalline cellulose (MCC); pregelatinized starch (PGS); Primogel (Sodium starch glycolate, USP/NF, Ph. Eur.); Primellose (Crosscarmelose sodium, USP/NF, ph. Eur.); di-calcium phosphate and tri-calcium phosphate. In one embodiment of the invention, the one or more bulking agents/binders are present from about 0.01% to about 30% of the total weight of the of the HCA-containing compound. In one embodiment of the invention, the one or more bulking agents/binders are present from about 0.1% to about 30% of the total weight of the of the HCA-containing compound. In one embodiment of the invention, the one or more bulking agents/binders are present from about 0.1% to about 25% of the total weight of the of the HCA-containing compound.

In one aspect of the invention, the HCA-containing compounds include HCA, one or more absorption-enhancer/controlled-release agents, one or more rate-controlling excipients, one or more lubricants, and one or more bulking agents/binders.

In one aspect of the invention, the HCA-containing compounds in include, HCA and one or more rate-controlling excipients. In one embodiment of the invention the HCA is present from about 1.0% to about 80% of the total weight of the HCA-containing compound. In one embodiment of the invention, the HCA is present from about 5% to about 70% of the total weight of the HCA-containing compound. In one embodiment of the invention, the HCA is present from about 10% to about 60% of the total weight of the HCA-containing compound. In one embodiment of the invention, the one or more rate-controlling excipients are present from about 0.0001% to about 60% of the total weight of the HCA-containing compound. In one embodiment of the invention, the one or more rate-controlling excipients are present from about 0.001% to about 50% of the total weight of the HCA-containing compound. In one embodiment of the invention, the one or more rate-controlling excipients are present from about 0.01% to about 25% of the total weight of the (−)-hydroxycitrate-containing compound.

In one aspect of the invention, the HCA-containing compounds in include, HCA and one or more lubricants. In one embodiment of the invention, the HCA is present from about 50% to about 99% of the total weight of the HCA-containing compound. In one embodiment of the invention, the HCA is present from about 50% to about 96% of the total weight of the HCA-containing compound. In one embodiment of the invention, the one or more lubricants are present from about 0.0001% to about 50% of the total weight of the of the HCA-containing compound. In one embodiment of the invention, the one or more lubricants are present from about 0.001% to about 50% of the total weight of the of the HCA-containing compound. In one embodiment of the invention the one or more lubricants are present from about 0.01% to about 50% of the total weight of the of the HCA-containing compound.

In one aspect of the invention, the HCA-containing compounds in include, HCA, one or more absorption-enhancer/controlled-release agents, and or more lubricants. In one embodiment of the invention, the HCA is present from about 1.0% to about 80% of the total weight of the HCA-containing compound. In one embodiment of the invention, the HCA is present from about 5% to about 70% of the total weight of the HCA-containing compound. In one embodiment of the invention, the HCA is present from about 10% to about 60% of the total weight of the HCA-containing compound. In one embodiment of the invention, the one or more absorption-enhancer/controlled-release agents are present from about 1.0% to about 50% of the total weight of the HCA-containing compound. In one embodiment of the invention, the one or more absorption-enhancer/controlled-release agents are present from about 1.0% to about 40% of the total weight of the HCA-containing compound. In one embodiment of the invention, the one or more absorption-enhancer/controlled-release agents are present from about 1.0% to about 30% of the total weight of the HCA-containing compound. In one embodiment of the invention, the one or more lubricants are present from about 0.0001% to about 10% of the total weight of the of the HCA-containing compound. In one embodiment of the invention, the one or more lubricants are present from about 0.001% to about 10% of the total weight of the of the HCA-containing compound. In one embodiment of the invention, the one or more lubricants are present from about 0.01% to about 5% of the total weight of the of the HCA-containing compound.

In one embodiment of the invention, the HCA-containing compound is included in a pharmaceutical composition containing a pharmaceutically-acceptable carrier.

In one aspect, the invention provides a method of suppressing the appetite in a subject, by administering to a subject in which appetite suppression is desired an HCA-containing compound of the invention in an amount sufficient to suppress the appetite in the subject.

In one aspect, the invention provides a method of reducing the cytoplasmic citrate lyase activity in a subject, by administering to a subject in which reducing cytoplasmic citrate lyase activity is desired an HCA-containing compound of the invention in an amount sufficient to reduce the citrate lyase activity.

In one aspect, the invention provides a method of increasing the fat metabolism in a subject, by administering to a subject in which increased fat metabolism is desired an HCA-containing compound in an amount sufficient to increase fat metabolism:

In one aspect, the invention provides a method of inducing weight-loss in a subject, by administering to a subject in which weight-loss is desired an HCA-containing compound in an amount sufficient to induce weight-loss.

In one aspect, the invention provides a method of reducing blood lipids and postprandial lipemia in a subject, by administering to a subject in which reduced blood lipids and postprandial lipemia is desired an HCA-containing compound in an amount sufficient to reduce blood lipids and postprandial lipemia.

### DETAILED DESCRIPTION OF THE INVENTION

### I. Definitions

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of an HCA-containing compound of the invention, as used herein, is a quantity sufficient to achieve a desired therapeutic and/or prophylactic effect, for example, an amount which results in the prevention of or a decrease in the symptoms associated with a disease, disorder or condition that is being treated, e.g., obesity, weight gain, hunger, hyperlipemia, postprandial lipemia. The amount of an HCA-containing composition of the invention administered to the subject will depend on the type and severity of the disease, disorder or condition, and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the HCA-containing compound of the invention sufficient for achieving a therapeutic or prophylactic effect will range from about 0.000001 mg per kilogram body weight per day to about 10,000 mg per kilogram body weight per day. In one embodiment, the dosage ranges are from about 0.0001 mg per kilogram body weight per day to about 100 mg per kilogram body weight per day. A common dosage range is between 1,000-5,000 mg per day. Another common dosage range is between 2,000-3,000 mg per day. A common daily dose is 3,000 mg per day. The HCA-containing compound of the invention can also be administered in combination alone, or with one or more additional therapeutic compounds.

### II. General

It is an object of the present invention to provide stable, non-hygroscopic HCA-containing compounds, e.g., potassium HCA, useful under those conditions necessary for tableting, encapsulation, the production of controlled-release vehicles and that can be incorporated into dry powders. Accordingly, the present invention teaches the use of absorption-enhancer/controlled-release agents and rate-controlling excipients for modifying the hygroscopic and other properties of HCA salts to stabilize and control the delivery of salts and derivatives of HCA. The invention provides methods to render non-hygrospcopic and stable, e.g., not prone to lactonization or acid-catalyzed degradation or sequestration by agents that inhibit their absorption or lead to their excretion, the otherwise hygroscopic salts of HCA in their relatively pure and active forms including, but not limited to potassium HCA salt, sodium HCA salt, and other HCA derivatives. The methods of the invention are useful to reduce the polar/ionic qualities of MCA salts and derivatives when presented to the intestinal lumen to provide advantages in absorption.

In one embodiment, the HCA-containing compounds of the invention include HCA, one or more absorption-enhancer/controlled-release agents and one or more rate-controlling excipients. The HCA can include, e.g., HCA free acid; HCA salts; HCA derivatives; or any combination thereof. In one embodiment, the HCA concentration is from about 1.0% to about 80% of the total weight of the HCA-containing compound. In one embodiment, the HCA concentration is from about 5% to about 70% of the total weight of the HCA-containing compound. In another embodiment, the HCA concentration is from about 10% to about 60% of the total weight of the HCA-containing compound.

The useful absorption-enhancer/controlled-release agents can include, but are not limited to, e.g., d-alpha-tocopheryl polyethylene glycol succinate (TPGS); Lubritab®; volcanic oils (e.g., such as glycerol monostearate, cetyl alcohol, stearyl alcohol); and/or various high viscosity grades of conjugated polyethylene glycol; ethylcellulose, carboxymethylcellulose, cellulose propionate (lower, medium or higher molecular weight), cellulose acetate propionate; cellulose acetate butyrate; cellulose acetate phthalate (CAP); cellulose triacetate; hydroxypropyl-methylcellulose phthalate; polymethyl methacrylate; polyethyl methacrylate; polybutyl methacrylate; polyisobutyl methacrylate; polyhexyl methacrylate; polyisodecyl methacrylate; polylauryl methacrylate; polyphenyl methacrylate; polymethyl acrylate; polyisopropyl acrylate; polyisobutyl acrylate; polyoctadecyl acrylate; polyethylene; polyethylene low density; polyethylene high density; polypropylene; polyethylene oxide; polyethylene terephthalate; polyvinyl isobutyl ether, polyvinyl acetate; polyvinyl acetate phthalate; polyvinyl chloride; polyurethane; other copolymers of acrylic and methacrylic and esters; waxes; shellac; zein (vegetable protein of the prolamine group); hydrogenated vegetable oils; polyvinyl alcohol; polyvinylpyrrolidone; methyl cellulose; hydroxypropyl cellulose; hydroxpropylmethyl cellulose or polyethylene glycol; or a mixture thereof. In one embodiment, the absorption-enhancer/controlled-release agent concentration is from about 1.0% to about 50% of the total weight of the HCA-containing compound. In another embodiment, the absorption-enhancer/controlled-release agent concentration is from about 1.0% to about 40% of the total weight of the HCA-containing compound. In yet another embodiment, absorption-enhancer/controlled-release agent concentration is from about 1.0% to about 10% of the total weight of the HCA-containing compound. In yet another embodiment, absorption-enhancer/controlled-release agent concentration is from about 2.0% to about 8.0% of the total weight of the HCA-containing compound.

The useful rate-controlling excipients can include, but are not limited to, e.g., polymers, plasticizers and disintegrants. The rate-controlling excipients can be hydrophobic. The rate-controlling excipients, e.g., plasticizers are useful to prevent the polymer shielding the HCA from becoming too brittle and cracking. The rate-controlling excipients are also useful to wick fluid into the matrix of the tablets, etc. The useful rate-controlling excipients can include, but are not limited to, e.g., Eastacryl® (dispersion of cellulose acetate pthalate); Kollicoat® IR (polyvinylalcohol-polyethyleneglycol graft-copolymer); cellulose acetate phthalate; Kollicoat® SR (polyvinylacetate dispersion stabilized with povidone and sodium laurylsulfate,); ethyl cellulose; Eudragit® (family of acrylate and methacrylate-based coatings); zein (vegetable protein); acrylic polymers; polyvinyl acetate phthalate; hydroxymethylpropylmethyl cellulose phthalate; cellulose acetate trimalleate; acrylic polymer plasticizers; polymers of polylactic acid; polymers of glycolic acid, and mixtures thereof; Primogel; Pruv™ (stearyl fumarate sodium); citrate esters; triethyl citrate; propylene glycol; and dibutyl sebacate. In one embodiment, the rate-controlling excipient concentration is from about 0.0001 to about 60% of the total weight of the HCA-containing compound. In one embodiment, the rate-controlling excipient concentration is from about 0.001% to about 50% of the total weight of the HCA-containing compound. In another embodiment, the rate-controlling excipient concentration is from about 0.01% to about 25% of the total weight of the HCA-containing compound.

Kollicoat® IR (polyvinylalcohol-polyethyleneglycol graft-copolymer) is an instant-release coating useful to create an HCA granulate composition for further processing that does not immediately become gummy when subjected to moisture and other challenges. Kollicoat® SR is a stabilized polyvinylacetate dispersion that provides a sustained-release coating. Eastacryl from Eastman is a dispersion of CAP used to provide a sustained-release coating.

In another embodiment, the HCA-containing compounds of the invention include HCA, one or more absorption-enhancer/controlled-release agents; one or more rate-controlling excipients; and one or more lubricants. A lubricant aids tablet manufacture by reducing friction in the tablet die during the act of compaction/compression and also during ejection. The lubricants improve powder flow characteristics, preventing the tablets from sticking to the punches, etc. Useful lubricants can include, but are not limited to, e.g., stearates (e.g., magnesium stearate, calcium stearate and sodium stearate, glycerol monostearate and stearic acid); Lubritab®; hydrogenated vegetable oils; waxes; talc; boric acid; sodium benzoate; sodium acetate; sodium chloride; DL-leucine; sodium oleate; sodium lauryl sulfate; magnesium lauryl sulfate and polyethylene glycols and kaolin. In one embodiment, the lubricant concentration is from about 0.0001 to about 10% of the total weight of the HCA-containing compound. In one embodiment, the lubricant concentration is from about 0.001% to about 10% of the total weight of the HCA-containing compound. In another embodiment, the lubricant concentration is from about 0.01% to about 5% of the total weight of the HCA-containing compound.

Lubritab® (hydrogenated vegetable oil, Type 1, NF; hydrogenated oil JP; hydrogenated oil JP; and hydrogenated vegetable oil, BP is made from fully hydrogenated refined vegetable oil that is sprayed into a dry, fine powder) is useful in the HCA-containing compounds of the invention as a lubricant. It is also useful as an auxiliary dry binder when tablets and capsules tend to cap or laminate. Lubritab® at up to 5% of the total weight of the HCA-containing compound can eliminate these problems and aid in producing satisfactory HCA-containing tablets. Lubritab® is more effective as a lubricant for HCA-containing compounds when added in the dry state in the last blending operation before compression and blending for 10-15 min. Lubritab® is useful as a lubricant in HCA-containing compounds of the invention when used in conjunction with an anti-adherent. An anti-adherent prevents the tablet from sticking to the tablet punch and to the die wall. Anti-adherents can include, but are not limited to, e.g., talc, corn starch, colloidal silicon dioxide, DL-leucine, sodium lauryl sulfate, and metallic stearates. Some ingredients, such as talc, can act in the same formulation as a lubricant, an anti-adherent and a glidant. A glidant improves the flow characteristics of the granulate. Glidants include, e.g., talc, corn starch and colloidal silicon dioxides, such as Aerosil™ (Degussa).

Furthermore, Lubritab® is useful in the HCA-containing compounds of the invention in controlled-release applications. In one embodiment, Lubritab® is used at 20-40% of the total weight of the HCA-containing compound. In another embodiment, Lubritab® is used at from about 5% to about 40% of the total weight of the HCA-containing compound. One skilled in the art will recognize that magnesium stearate, other stearates, hydrogenated vegetable oils and related compounds similarly can be adapted to the purpose of controlling the release of HCA salts and compounds.

In another embodiment, the HCA-containing compounds of the invention include HCA, one or more absorption-enhancer/controlled-release agents; one or more rate-controlling excipients; and one or more bulking-agents/binders. These bulking-agents/binders are also useful to modulate the HCA release rate. Useful bulking-agents/binders include, but are not limited to, e.g., starch paste; acacia; sucrose; poly vinyl pyrrolidone (PVP); hydroxy proplyl methyl cellulose (HPMC); methyl cellulose; and gelatin. In one embodiment, water-wicking agents, such as microcrystalline cellulose, are used in the HCA-containing compound of the invention to regulate how fast a controlled-release tablet is penetrated when it reaches a high pH region. In another embodiment disintegrants are useful as bulking agents in the HCA-containing compounds of the invention. Useful disintegrants include, but are not limited to, e.g., potato starch; micro crystalline cellulose (MCC); pregelatinized starch (PGS); Primogel (Sodium starch glycolate, USP/NF, Ph. Eur.); Primellose (Crosscarmelose sodium, USP/NF, ph. Eur.)

The useful bulking-agents/binders can include, but are not limited to, e.g., di-calcium phosphate and tri-calcium phosphate. In one embodiment, the bulking agent/binder concentration is from about 0.01% to about 30% of the total weight of the HCA-containing compound. In one embodiment, the bulking agent/binder concentration is from about 0.1% to about 30% of the total weight of the HCA-containing compound. In another embodiment, the bulking agent/binder concentration is from about 0.1% to about 25% of the total weight of the HCA-containing compound.

In yet another embodiment, the HCA-containing compounds of the invention include HCA, one or more absorption-enhancer/controlled-release agents; one or more rate-controlling excipients; one or more lubricants; and one or more bulking-agents/binders.

in yet another embodiment, the HCA-containing compounds of the invention include HCA and one or more rate-controlling excipients.

In yet another embodiment, the HCA-containing compounds of the invention include HCA and one or more lubricants.

In yet another embodiment, the HCA-containing compounds of the invention include HCA, one or more absorption-enhancer/controlled-release agents; and one or more lubricants.

In another embodiment, the aforementioned HCA-containing compounds of the invention have chloride content of less than about 2.5% weight. In one embodiment, the chloride content of the HCA-containing compound of the invention is less that about 1.0% weight. In yet another embodiment, the chloride content of the HCA-containing compound of the invention is less than about 0.5% weight.

In yet another embodiment, the aforementioned HCA-containing compounds of the invention have a total halogen content as chloride of less than about 2.9% weight. In one embodiment, the HCA-containing compounds of the invention have a total halogen content as chloride of less than about 1.0% weight. In yet another embodiment, the HCA-containing compounds of the invention have a total halogen content as chloride of less than about 0.6% weight.

In one embodiment, the HCA-containing compounds of the invention are included in a dry delivery system, e.g., tablet, dry powder, and dry meal replacement mixture. In another embodiment, the HCA-containing compounds of the invention are included in a liquid delivery system, e.g., capsule, caplet, or beverage. In yet another embodiment, the HCA-containing compounds of the invention are used in controlled-release vehicles, e.g., tablet, caplet, and capsules.

The present application is related to U.S. Pat. No. 6,447,807, issued Sep. 10, 2002, the contents of which are hereby incorporated by reference in its entirety.

### III. Characteristics of HCA and HCA Salts

Early work ascribed the weight loss benefit to HCA, its salts and its lactone form. See generally, U.S. Pat. No. 3,764,692 granted to John M. Lowenstein. One commonly offered explanation for the biological and therapeutic effects of HCA is the inhibition of cytoplasmic (cytosolic) ATP-citrate lyase (D. Clouatre and M. E. Rosenbaum, The Diet and Health Benefits of HCA (Hydroxicitric Acid), 1994). In subsequent studies the lactone form of HCA was shown to be far less effective than the sodium salt form of HCA for weight loss purposes, in part because the lactone form lacks the proper affinity for ATP-citrate lyase, known to be a target of the actions of HCA (Lowenstein and Brunengraber, Methods Enzymol. 1981; 72:486-97). Under conditions that promote lactonization (e.g., acidic conditions), free HCA undergoes rapid inactivation. Indeed, inclusion of currently available mineral salts of HCA in a prepared beverage of acidic pH leads to the development of HCA lactone over time.

The use of free HCA concentrate in food products has been described in U.S. Pat. No. 5,536,516, but it does not teach any particular advantage for the use of HCA in weight loss or for other medicinal purposes. Even brief exposure of the potassium and sodium salts of HCA to acidic conditions or flavored beverages results in chemical changes in these HCA salts. In some cases the beverages actually change color upon addition of potassium HCA or sodium HCA salts.

Free HCA is extremely ionic and does not pass readily through the gut membrane. The free acid form of HCA can be sequestered by binding soluble and insoluble fibers as well as by many other compounds, thus rendering HCA biologically unavailable. There is evidence that the free HCA and HCA lactone are both irritating to the gastrointestinal tissues if consumed regularly in large amounts.

Generally, calcium HCA and magnesium HCA salts, either alone or in the form of various mixtures together, or in combination with the potassium HCA and sodium HCA salts, are not preferred delivery forms for HCA. Calcium HCA and magnesium HCA salts are also not readily absorbed across the gastrointestinal tract because they are poorly soluble in aqueous media. These HCA salts are also reactive with bile acids and fats in the gut and/or are sequestered by binding to soluble and insoluble fibers or other substances in the diet or secreted during digestion (Heymsfield, Steven B, et al. JAMA 1998; 280(18): 1596-1600; Letters, JAMA 1999; 282: 235). For example, the action of stomach acid may free one of the two valences of calcium HCA or magnesium HCA salts for attachment to fats, bile acids, gums, fibers, pectins, and so forth and so on, which is an undesirable outcome. The addition of small amounts of magnesium HCA to potassium HCA, however, improves the transit of potassium HCA across cell membranes. By contrast, calcium, impedes the transit of potassium HCA across cell membranes.

Calcium/potassium HCA (Super CitriMax®) is not well absorbed as only 20% of the dose ingested by fasted subjects was detected in the blood using gas chromatography/mass spectroscopy technique (Loe of al., Anal Biochem. 2001, 1; 292(1): 148-54). Loe and coworkers reported that the absorption of calcium/potassium HCA (Super CitriMax®) peaked 2 hours after administration, and that the compound remained in the blood for more than 9 hours after ingestion (Loe et a, FASEB Journal, 15 4:632, Abs. 501.1, 2001). Eating a meal shortly after taking Super CitriMax® reduced its absorption by about 60%. Moreover, animal trials (see U.S. Pat. No. 6,476,071) have further demonstrated that in order for the potassium salt to be maximally effective, the ligand must be fully bound to the HCA with only trivial amounts of contaminants, including most other minerals or fibers or sugars.

Calcium HCA salt has some further disadvantages that may limit its therapeutic use. Calcium uptake from the gut is highly regulated and under normal circumstances does not exceed approximately 35% of that found in foods and supplements. The uptake of calcium declines as the dosage of calcium is increased. This may limit the use of calcium HCA where large doses may need to be ingested. For example, for weight loss and other purposes, a minimally effective amount of HCA derived from its calcium salt requires the administration of between 12 and 15 grams of a 50% material. This amount of calcium HCA may lead to undesirably elevated levels of binding and excretion of other dietary minerals, such as zinc, aside from presenting difficulties in administration.

HCA sodium salt has disadvantages for long-term administration to a subject. First, sodium HCA lacks positive metabolic effects with regard to obesity. Second, sodium HCA has potential hypertensive actions. Indeed, several of the early Indian-supplied “potassium” salts were, in fact, mixtures of calcium, potassium and sodium (−)-hydroxycitrate. The amount of sodium in these HCA preparations exceeded that allowed in low sodium diets notwithstanding the fact that added sodium is ill-advised in any modern diet. In contrast, potassium HCA does not possess the disadvantages associated with sodium HCA.

A preferred salt of HCA for pharmaceutical use is potassium HCA. The mineral potassium is fully soluble, as is its HCA salt, and is known to possess cell membrane permeability which is 100 times greater than that possessed by sodium. However, the potassium salt of HCA, as is also true of the sodium salt, is extremely hygroscopic and thus not suitable under normal circumstances for the production of dry delivery forms. In drawing moisture to itself, potassium HCA will also tend to bind to available binding sites of compounds in its immediate environment, and this action often later will markedly impede the assimilation of potassium HCA from the gut. Potassium HCA is also not suitable for liquid delivery forms inasmuch as potassium HCA in solution will slowly lactonize to an equilibrium which is dependent upon the pH.

### IV. Select HCA-Containing Compounds and their Delivery

Several international patent applications and U.S. patents disclose HCA-containing compounds and its delivery as calcium, magnesium and admixtures of salts. International patent application WO 99/03464, filed 28 Jan. 1999, is directed to HCA-containing compounds with 14 to 26 wt % calcium HCA, and approximately 24 to 40 wt % potassium HCA or approximately 14 to 24 wt % sodium HCA, or a mixture thereof, each calculated as a percentage of the total HCA content of the composition for use in dietary supplements and food products. Studies assessing such a composition showed that its assimilation is exceedingly poor even when taken on an empty stomach (Loe et al., Anal Biochem. 2001 May 1; 292(1): 148-54) and that eating a meal shortly after taking it reduced its absorption by about 60% (Loe et al., Time Course of Hydroxycitrate Clearance in Fasting and Fed Humans, FASEB Journal, 15, 4: 632, Abs. 501.1, 2001). Further, studies comparing the effect of various HCA-containing compounds on body weight and food intake in a rat obesity model showed that a test composition of calcium/potassium HCA salt identical to that described by WO 99/03464 was inferior compared to potassium HCA salt in reducing weight gain in middle-aged rats fed a 30% fat diet (see U.S. Pat. No. 6,476,071 B1). Specifically, at the level of intake used experimentally on a 30% fat diet, potassium HCA increased protein as a percentage of body weight while reducing fat as a percentage of body weight. In contrast, the calcium/potassium salt HCA test composition increased fat and reduced protein as percentages of body weight.

International patent application WO 00/15051 is directed to a method of making calcium HCA more soluble by under-reacting the material, i.e., leaving a substantial amount of HCA lactone in the finished product. This procedure, however, does little to improve the uptake of HCA. The problems with HCA lactone are discussed above, and the HCA lactone in large amounts is known to be irritating (Ishihara et al., J Nutr. 2000 December; 130(12): 2990-5). Making calcium soluble, again, does nothing to prevent its reactivity with compounds in the gut, e.g., bile salts, or to improve the general rate of assimilation of calcium HCA. It is noteworthy that the process disclosed in WO 00/15051 was previously disclosed by others in 1997 (Sawada et al., Journal of Japan Oil and Chemicals/Nihon Yukagaku Kaishi 1997 December; 46, 12: 1467-1474) and many months earlier in Japanese.

International patent application WO 02/014477 is directed to a composition comprising HCA in combination with either one or both of garcinol and anthocyanin. Garcinol is a common contaminant of HCA products, and thus, it is typically present in the salts which have been used for other clinical studies, i.e., extracts rather than synthesized pure HCA salts. It is unknown whether the additive effect shown in WO 02/014477 extends beyond the mild response reported if higher dosages of either component are ingested. However, a published paper examining the impact of flavonoids derived from Garcinia cambogia found that a dose response study revealed biphasic activity. Higher doses were less effective in reducing lipid levels in serum and tissues, although devoid of toxic effects. (Koshy A S, Vijayalakshmi N R. Impact of certain flavonoids on lipid profiles—potential action of Garcinia cambogia flavonoids. Phytother Res. 2001 August; 15(5):395-400.)

U.S. Pat. No. 6,221,901 is directed to the preparation and uses of magnesium HCA. The high dosage of magnesium HCA required to achieve the indicated results, however, may limit therapeutic utility of the composition. For example, in order to achieve a hypotensive effect, for instance, the inventors fed their animals 500 mg/kg magnesium HCA. Using the standard 5:1 multiplier for rat to human data, the dose of magnesium hydroxycitrate employed by Shrivastava at al. is equivalent to a human ingesting 100 mg/kg/day or 7 grams for the average-sized human subject. Of this amount, 45% would be elemental magnesium; hence resulting in a human ingesting the equivalent of approximately 3.15 grams of magnesium. The Recommended Dietary Allowances, 10th edition (National Research Council, 1989), indicates that most humans begin to suffer diarrhea at more than 350 mg/day. In other words, the test dose used by Shrivastava at al, is nearly 10 times the dose at which side effects would normally be expected to begin to appear. The induced diarrhea itself would lower blood pressure rapidly.

U.S. Pat. No. 5,783,603 is directed to a technique for the production of potassium HCA. The potassium HCA prepared by this method requires that the milling, sifting, blending and packing of the potassium HCA be carried out in a nitrogen atmosphere as the potassium HCA preparation is otherwise hygroscopic. That is, if left in the open air outside of a humidity-controlled environment, the potassium HCA produced according to that patented method will begin to absorb moisture within a few min. This property will limit the use of this material as a component of dry pharmaceutical or nutraceutical preparations. There are available low-pH versions of potassium HCA, i.e., pH of between 7 and 8, but such forms of potassium hydroxycitrate are under-reacted, infused with lactone, or suffer similar failings which make them inferior in the physiological effects to the properly prepared product. A fully reacted potassium HCA will have a pH greater than 9.

U.S. Pat. No. 6,447,807 is directed to methods for making the hygroscopic salts of HCA workable and for controlling the delivery of HCA salts. The methods of the present invention are distinct from the methods of the issued patent as they teach the use of TPGS. The use of TPGS in the preparation of HCA-containing compounds improves upon the methods of U.S. Pat. No. 6,447,807 by reducing or eliminating both the need to spray-dry HCA onto a separate carrier, e.g., maltodextan and steps requiring special spray or freeze drying of the HCA-containing compound.

### V. HCA Delivery

The effective delivery of HCA to a subject in need thereof has been limited by the few methods for producing a controlled-release form of HCA, regardless of the salt used. Tests performed to establish the appetite-suppressing effects of HCA demonstrated that a single large oral dose or two divided oral doses totaling one fourth the size of the single dose resulted in a 10% or greater reduction in food consumption in experimental animals fed a high-sugar diet. This result continued over many weeks with the chronic ingestion of HCA. The requirement for at least two divided doses of HCA for efficacy is the only thoroughly established procedure to date.

Giving HCA as multiple doses, as is true of any drug, is inconvenient and is not supported by good patient compliance. Multiple doses given in the form of any of the current salts is also wasteful in that any material delivered to the body which is above the baseline or threshold necessary to produce benefits is simply an excess which is excreted. Controlled release of HCA avoids both excess and waste, on the one hand, and gaps in coverage, on the other hand. Controlled release makes it possible to simplify the dosage schedule to one daily administration. Moreover, it is to be expected that a smaller amount of HCA delivered by controlled release will provide benefits which are superior to those found with a larger amount of HCA supplied after a normal fashion in at least two dosages.

As noted above, the potassium salt of HCA is the most efficacious form of HCA to be used for human weight loss and for other pharmaceutical and/or nutraceutical purposes, followed secondarily for these purposes by the sodium salt. The potassium and the sodium salts of HCA present very similar difficulties in handling and manipulation. Potassium HCA is extremely hygroscopic and tends to bind with water in the open air to form a non-palatable paste not suitable for use in tablets, capsules or powders. This material can be admixed with orange juice or water, but requires vacuum pouch sealing under a humidity-controlled atmosphere and is inconvenient for the patient to use. Potassium HCA is reactive with a large number of compounds (tannins, gums, fibers, pectins, and so forth) are thereby readily suffers large losses in pharmacological availability.

### VI. Granulation of HCA-Containing Compounds and the Use of TPGS

It is a known pharmaceutical practice to cover material to be granulated, especially hygroscopic materials, with molten oils such as hydrogenated vegetable oil, glycerol monostearate, cetyl alcohol, stearyl alcohol and various high viscosity grades of conjugated polyethylene glycol. The use of TPGS in the HCA-containing compounds was not previously known.

TPGS has a melting point of 40° C. and is as water soluble as polyethylene glycol. TPGS is synthesized by esterifying d-alpha-tocopheryl succinate with polyethylene glycol (PEG) 1000 (i.e., the molecular weight of PEG 1000 is approximately 1,000 daltons). The resulting product is a pale yellow, waxy solid substance that is amphipathic and hydrophilic with a molecular weight of approximately 1,513 daltons. d-alpha-Tocopherol comprises 26% of TPGS. TPGS is variously known as d-alpha-tocopheryl polyethylene glycol 1000 succinate and d-alpha-tocopheryl d-alpha-tocopheryl PEG 1000 succinate. Inasmuch as there are eight stereoisomers of alpha-tocopherol, more complete chemical names for TPGS include RRR-alpha-tocopheryl polyethylene glycol 1000 succinate, 2R,4′R, 8′R-alpha-tocopheryl polyethylene glycol 1000 succinate and 2,5,7,8-tertramethyl-2-(4′,8′,12′-trimethyltridecyl)-6-chromanyl polyethylene glycol 1000 succinate. PDRhealth, an online-component of the Medical Economics Company (see http://www.gettingwell.com/drug_info/nmdrugprofiles/nutsupdrugs/alp_{—}0091.shtml, which provides a description of the pharmacokinetics of TPGS). It is anticipated that, in the future, other isomers of tocopherol will become available for the uses proposed here as natural extensions of the art. Such extensions of the art are contemplated to be within the scope of the present invention.

TPGS has the capability to act as an emulsifying agent in the formulation of organic water-based emulsions and can be used as a molten direct spray on certain products that have low bioavailability. The product has an HLB (hydrophile/lipophile balance) of ˜13. It is stable to air, but reacts with alkali. TPGS can serve as an excellent coating for granulated material or oils which have low intestinal absorption. TPGS also has benefits over many other chemical non-nutritive/non-natural emulsifiers.

The product is structurally similar to an amphiphile. It has a dual nature, with part of the molecule comprising the hydrophilic polar head and the other liphophilicity. The exact portion of the molecule comprising the hydrophilic or polar end head or the lipophilic alkyl tail cannot be elucidated from the molecular structure. The generally accepted view is that the polyethylene glycol portion serves as the hydrophilic polar head while the tocopheryl succinate portion serves as the lipophilic tail. TPGS provides vitamin E at 387-447 IU/g. This material is melted using a hot plate or other device and stirred with a magnetic stirring rod at a temperature of approximately 40° C. or higher. It is then sprayed onto the material to be granulate in a fluid bed dryer using an inlet temperature of approximately 30° C. and the spray is adjusted so as to place a fine mist over the material to be granulated. As soon as the powder is thoroughly blended with the molten phase and dried to a hard solid surface, a second coat of hydrogenated oil is applied.

This overcoat of solid oil is preferably molten hydrogenated vegetable oil. This material is purely lipophilic and has little or no amphiphilic character to its nature. It is made into a molten phase by heating and stirring while spraying onto the powder with previously granulated TPGS. Over these two oil layers is sprayed and dispersed the rate-controlling polymer or polymers.

TPGS improves the uptake of cyclosporin and many other compounds. Vitamin E TPGS is also used in the solvent extraction/evaporation technique for fabrication of polymeric nanospheres of an antineoplastic drug Paclitaxel (Taxol®) for cancer chemotherapy (BED-Vol. 50, 2001 Bioengineering Conference ASME 2001). The hypothesis that HCA-containing compounds may benefit from the self-micelle-forming properties of TPGS led to studies assessing the effect of TPGS on the stability and hygroscopic nature of HCA-containing compounds. Studies assessing the effect of formulating HCA-containing preparations with TPGS demonstrated that TPGS is especially well-suited for granulation of HCA-containing compounds and enhances their bioavailability. That is, the inventors have been successful in granulating the potassium salt form of HCA into a workable powder. This workable powder can be further manipulated according to the procedures taught in the U.S. Pat. No. 6,447,807 to produce a product formulated for controlled delivery. The same results can be extended to sodium and other salts of HCA and their mixtures.

As noted above, U.S. Pat. No. 6,447,807 is directed to methods for making the hygroscopic salts of HCA workable and for controlling the delivery of HCA salts. The methods of the present invention are distinct from the methods of the issued patent as they teach the use of TPGS. The use of TPGS in the preparation of HCA-containing compounds improves upon the methods of U.S. Pat. No. 6,447,807 by reducing or eliminating both the need to spray-dry HCA onto a separate carrier, e.g., maltodextan and steps requiring special spray or freeze drying of the HCA-containing compound. The present invention can substitute fluid bed drying for these latter processes.

### VII. HCA-Containing Compounds

HCA-containing compounds of the invention which include, but not limited to, e.g., HCA free acid, HCA salts, HCA derivatives, or any combination thereof, to make a granulate which can be used alone or further formulated with pharmaceutically acceptable compounds, vehicles, or adjuvants with a favorable delivery profile, i.e., suitable for delivery to a subject. Such compositions typically comprise the HCA-containing compound of the invention and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. The pH can be adjusted with acids or bases, such as hydrochloric acid or sodium hydroxide.

Oral compositions generally include an inert diluent or an edible carrier. They can be enclosed in gelatin capsules, caplets or compressed into tablets. For the purpose of oral therapeutic administration, the HCA-containing compound of the invention can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

The HCA-containing compound of the invention can also be prepared as pharmaceutical compositions in the form of suppositories (e.g., with conventional suppository bases such as cocoa butter and other glycerides) or retention enemas for rectal delivery.

In one embodiment, the HCA-containing compounds of the invention are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the HCA-containing compound and the particular therapeutic effect to be achieved, and the limitations inherent in the art of compounding such an active compound for the treatment of individuals.

The pharmaceutical compositions can be included in a container, pack, or dispenser together with instructions for administration.

### VIII. Methods of Preparing HCA-Containing Compound Using TPGS

TPGS can be applied to a dry HCA preparation including, but not limited to, e.g., HCA free acid, HCA salts, HCA derivatives, or any combination thereof, to make a granulate which can be used alone or further formulated with pharmaceutically acceptable compounds, vehicles, or adjuvants with a favorable delivery profile, i.e., suitable for delivery to a subject. (−)-Hydroxycitric acid and its lactone, which are liquids, can be made amenable for employment in this invention by first being laid upon a suitable desiccant, e.g., fumed silicon dioxide, as taught in U.S. Ser. No. 10/303,117 (Clouatre, Clouatre and Dunn), in which examples include liquid potassium HCA. The HCA preparations of the invention may be administered to a subject in need thereof by any suitable route, including, but not limited to, e.g., oral, intraperitoneal, and intravenous. In one embodiment the HCA preparation of the invention is administered to a subject one or more times a day. In one embodiment, the HCA preparation of the invention is administered to a subject once a day.

HCA, HCA salts and HCA derivatives can be prepared as conjugates with lipids, the primary agent being TPGS. Further preparation with time-released polymers, when compounded as a controlled release tablet or capsule, provides prolonged dwell time in the body after oral administration. Mucosal adhesive and similar agents can also be employed. The amount of TPGS will normally range between 2% and 10% of the finished product. A similar range will be typical for hydrogenated vegetable oils or similar items used to complement the actions of the TPGS. Methods for the preparation of HCA-containing compounds of the invention are detailed in Examples 1 through Example 4.

Briefly, HCA, an HCA salt or a combination of HCA salts are blended in a low humidity environment with TPGS to yield a TPGS/HCA mixture. The TPGS/HCA mixture is further blended with molten oils, such as hydrogenated vegetable oil, glycerol monostearate, cetyl alcohol, stearyl alcohol and/or various high viscosity grades of conjugated polyethylene glycol to yield a crude TPGS/HCA granulate mixture. The crude TPGS/HCA granulate mixture is then blended with a polymer wherein the polymer to yield an HCA-containing compound of the invention. The polymer film should have enteric properties as taught in U.S. Pat. No. 6,447,807. Suitable polymers include, but are not limited to, e.g., cellulose acetate phthalate, ethyl cellulose, Eudragit L55®, zein, acrylic polymers, hydroxymethylpropylmethyl cellulose phthalate, polyvinyl acetate phthalate, cellulose acetate trimalleate, acrylic polymer plasticizers, polymers of polylactic acid, polymers of glycolic acid, and mixtures thereof. The HCA-containing compound of the invention is then formulated into tablets, capsules, prepared dry drink mixes, prepared liquid drinkable products and edible bars.

In one embodiment, the TPGS is admixed with the other components of the composition from about 1.0% to about 50% by weight of the amount of HCA on a dry weight basis. In one embodiment, the TPGS is admixed with the other components of the composition from about 1.0% to about 20% by weight of the amount of HCA on a dry weight basis. In another embodiment, the TPGS is admixed with the other components of the composition from about 2% to about 10% by weight of the amount of HCA on a dry weight basis.

### IX. Uses of the HCA-Containing Preparation of the Present Invention

A. Prophylactic and Therapeutic Uses of the HCA-Containing Compounds

The HCA-containing compounds of the present invention are useful in potential prophylactic and therapeutic applications implicated in a variety of disorders, diseases and conditions in a subject including, but not limited to, e.g., obesity, overweight, hunger, deficiencies in fat metabolism, hyperlipemia, and postprandial lipemia (i.e., the level of lipids in the blood following a meal). By way of a non-limiting example, the compositions of the invention will have efficacy for treatment of subjects suffering from the disorders mentioned in the Diseases and Disorders, infra.

B. Determination of the Pharmacokinetics or Biological Effect of the HCA-Containing Compounds

The pharmacokinetics of HCA-containing compounds, including absorption, can be determined by measuring the HCA level in the blood of subjects administered an HCA-containing compound using gas chromatography/mass spectroscopy technique (Loe et al., Anal Biochem. 2001, 1; 292(1):148-64) and as further detailed by Loe et al., (FASEB Journal, 2001, 15 4:632, Abs. 501.1). The assessment and comparison of the pharmokinetics of test compounds is well known in the art.

The effect of HCA-containing compounds on the activity of ATP-citrate lyase can be measured using the ATP-citrate lyase assay procedure as detailed by Houston and Nimmo (Biochim Biophys Acta 1985 Feb. 21; 844(2): 233-9). A reduction in ATP-citrate lyase activity in the presence of HCA-containing compound when compared to the level of ATP-citrate lyase activity observed in the absence of HCA-containing compound indicates that the HCA-containing compound inhibits ATP-citrate lyase enzyme.

In various embodiments of the invention, suitable in vitro or in vivo assays are performed to determine the effect of a specific HCA-based therapeutic and whether its administration is indicated for treatment of the affected tissue in a subject.

In various specific embodiments, in vitro assays can be performed with representative cells of the type(s) involved in the patient's disorder, to determine if a given HCA-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

C. Diseases, Disorders and Conditions

The invention provides for both prophylactic and therapeutic methods of treating a subject at risk of (or susceptible to) a disease or having a disorder associated with lipid metabolism, e.g., but not limited to, obesity, overweight, deficiencies in lipid metabolism, hyperlipemia, postprandial lipemia, disorders where inhibition of inhibit cytoplasmic citrate lyase is advantageous or physical conditions such as hunger.

The HCA-containing compounds of the present invention are useful prevent or treat diseases, disorders or conditions where inhibition of inhibition of ATP-citrate lyase is advantageous, e.g., reduction of cholesterol level. Berkhout et al., (Biochem J. 1990 Nov. 15; 272(1): 181-6) studied the effect of HCA on the activity of the low-density lipoprotein receptor and 3-hydroxy-3-methylglutaryl-CoA reductase levels in the human hepatoma cell line Hep G2. After 2.5 h and 18 h incubations with HCA at concentrations of 0.5 mM or higher, incorporation of [1,5-14C]citrate into fatty acids and cholesterol was strongly inhibited. It was concluded that this decrease reflected an effective inhibition of ATP citrate-lyase. Cholesterol biosynthesis was decreased to 27% of the control value as measured by incorporations from ³H₂O, indicating a decreased flux of carbon units through the cholesterol-synthetic pathway.

The HCA-containing compounds of the present invention are useful to prevent or treat diseases or disorders associated with lipid metabolism, e.g., but not limited to, obesity; overweight; hyperlipemia; postprandial lipemia; and deficiencies in lipid metabolism, e.g., insulin resistance. Ishihara et al., (J Nutr. 2000 December; 130(12): 2990-5) studied the effect of chronic HCA administration on both carbohydrate utilization and lipid oxidation. The respiratory exchange ratio of test subjects was significantly lower in the HCA group during both resting and exercising conditions. These results suggest that chronic administration of HCA promotes lipid oxidation and spares carbohydrate utilization in test subjects at rest and during running.

Under conditions that elevate de novo lipogenesis in humans, HCA reduced fat synthesis and increased energy expenditure (Kovacs and Westerp-Plantenga, Society for the Study of Ingestive Behavior, Annual Meeting, 2001, Abstr. page 27). The HCA-containing compounds of the present invention, therefore, are useful in diseases or disorders associated with lipid metabolism.

The HCA-containing compounds of the present invention are useful to prevent or treat hunger and to promote satiety in a subject as the administration of HCA to subjects has been reported to promote appetite suppression and satiety (Westerterp-Plantenga and Kovacs, Int. J. Obes. Relat. Metab. Disord., 2002, 26(6): 870-2).

### EXAMPLES

The disclosures of these references provided in this list immediately above are herein incorporated in their entirety by reference thereto.

### Example 1

A formulation of the following composition (see Table 1) was prepared:

TABLE 1

| | | Amount | |
|---|---|---|---|
| Item # | Ingredient | (mg/Tablet) | Percent |
| 1 | HCA calcium salt | 500 | 71.43 |
| 2 | Microcrystalline cellulose | 17 | 2.42 |
| 3 | Dicalcium phosphate | 45 | 6.42 |
| 4 | Corn starch | 9 | 1.28 |
| 5 | TPGS | 46 | 6.60 |
| 6 | Hydrogenated vegetable oil | 50 | 7.14 |
| 7 | Cellulose acetate phthalate | 15 | 2.14 |
| 8 | Carbopol ® 974P Carbomer | 15 | 2.14 |
| 9 | Magnesium Stearate | 3 | 0.43 |
| | TOTAL | 700 | 100 |

The method of preparation was as follows:
1. Items #1-4 were weighed and blended in a fluid bed dryer for 4-5 min. Item #5 was then dissolved by heating to 40° C. until molten, and stirred with a magnetic stir rod. After the powders were blended, steady blending was continued while adding the TPGS (item #5) as a molten liquid. The TPGS was poured in fluid until an even granulate was formed. Next, the hydrogenated vegetable oil was melted until molten and fluid in nature. This material was then sprayed, while at the same time stirring with a magnetic stir rod. Blending with air at 30° C. was continued. When all the material was thoroughly coated and the granulate was hardened, the cellulose acetate phthalate, which had been completely dissolved in ammoniated water, was sprayed. Spraying was continued until all the granulate had been covered, then allowed to dry at room temperature in the fluid bed dryer with continuous blending. When the granulate was dry, it was removed from the bowl, and passed through a #093 screen using a D3 Fitzmill comminutor.
2. When the granulate was dried and reduced in size, it was blended in fluid bed first with Carbopol-974P. When completely blended, magnesium stearate was added and blended for 2-3 min.
3. The mixed granulate was then placed on a rotary press and compressed into tablets with a weight of 700 mg and a fracture force of 10-15 kg.

These tablets resisted disintegration for at least about 1 h with stirring in monophosphate buffer solution, pH 6.8. The phosphate buffer, pH 6.8 used during simulated intestinal disintegration was made using potassium phosphate, monobasic (34.02 g in 5 L water), and adjusting to the solution to pH 6.8 using 1N sodium hydroxide. Also, there was no evidence of breakdown upon exposure with stirring to simulate gastric fluid without pepsin (HCl solution with pH 1.2) for 60 min. Inasmuch as the tablet typically will leave an empty stomach within this period of time, this was considered to be adequate. This compares favorably with previous formulations of non-enteric capsules containing non-enteric HCA that disintegrated in stomach fluids within 15-30 min and tablets usually within the same period of time and always within less than 60 min. For some purposes, 55 min under a pH of 6.8 is ideal for the time-release of HCA from an HCA-containing compound, whereas longer time-release formulations are preferred for once-per-day dosing of an HCA-containing compound.

### Example 2

A formulation of the following composition (see Table 2) was prepared:

TABLE 2

| | | Amount | |
|---|---|---|---|
| Item # | Ingredient | (mg/Tablet) | Percent |
| 1 | HCA potassium/calcium salt | 500 | 64.93 |
| 2 | Dicalcium phosphate | 50 | 6.49 |
| 3 | Microcrystalline cellulose | 30 | 3.90 |
| 4 | Corn starch | 5 | 0.65 |
| 5 | TPGS | 30 | 3.90 |
| 6 | Hydrogenated cotton seed oil | 60 | 7.79 |
| 7 | Cellulose acetate phthalate | 30 | 3.90 |
| 8 | Carbopol ® 974-P carbomer | 60 | 7.79 |
| 9 | Magnesium stearate | 5 | 0.65 |
| | TOTAL | 770 | 100 |

The method of preparation was as follows:
1. Items #1-4 were weighed and blended in a fluid bed dryer for 4-5 min. Item #5 was then dissolved by heating to 40° C. until molten, and stirred with a magnetic stir rod. After the powders were blended, steady blending was continued while adding the TPGS (item #5) as a molten liquid. The TPGS was poured in fluid until an even granulate was formed. Next, the hydrogenated vegetable oil was melted until molten and fluid in nature. This material was then sprayed, while at the same time stirring with a magnetic stir rod. Blending with air at 30° C. was continued. When all the material was thoroughly coated and the granulate was hardened, the cellulose acetate phthalate, which had been completely dissolved in ammoniated water, was sprayed. Spraying was continued until all the granulate had been covered, then allowed to dry at room temperature in the fluid bed dryer with continuous blending. When the granulate was dry, it was removed from the bowl, and passed through a #093 screen using a D3 Fitzmill comminutor.
2. When the granulate was dried and reduced in size, it was blended in fluid bed first with Carbopol-974P. When completely blended, magnesium stearate was added and blended for 2-3 min.
3. The mixed granulate was then placed on a rotary press and compressed into tablets with a weight of 700 mg and a fracture force of 10-15 kg.

The disintegration of this novel stable HCA formulation, which was assessed in pH 6.8 monophosphate buffer, was 3-4 hours. This compares favorably with previous formulations of non-enteric capsules containing non-enteric HCA that disintegrated in stomach fluids within 15-30 min and tablets usually within the same period of time and always within less than 60 min.

### Example 3

### Slow-Release HCA Formulation

In one embodiment of the present invention, methacrylate polymer was used as the film retardant. In the present example, Eudragit® was the polymer used as a non pH-sensitive covering with the bioadhesives.

Eudragit® RS is available as a powder or a 30% aqueous dispersion. This methacrylate powder or solution is impermeable to water. Drugs entrapped in its matrix diffuse out by passive diffusion, regardless of the pH. It had a sticky component in pharmaceutical mixtures and therefore required the use of ancillary agents, such as triethyl citrate, talc, and/or magnesium stearate.

An example of this formulation in the use of slow release of HCA was prepared according to the following composition (see Table 3):

TABLE 3

| | | Amount | |
|---|---|---|---|
| Item # | Ingredient | (mg/Tablet) | Percent |
| 1 | HCA potassium salt | 750 | 76.14 |
| 2 | Microcrystalline cellulose | 20 | 2.03 |
| 3 | Dicalcium phosphate | 87 | 8.83 |
| 4 | Corn Starch | 9 | 0.91 |
| 5 | TPGS | 35 | 3.55 |
| 6 | Hydrogenated vegetable oil | 35 | 3.55 |
| 7 | Eudragit ® 30% RS | 15 | 1.52 |
| 8 | Triethyl citrate | 5 | 0.51 |
| 9 | Talc | 10 | 1.02 |
| 10 | Carbopol ® 974P carbomer | 16 | 1.62 |
| 11 | Magnesium stearate | 3 | 0.30 |
| | TOTAL | 985 | 99.98 |

The method of preparation was as follows:
1. The potassium HCA salt was thoroughly dried before use and the environment for preparation was low humidity. In a fluid bed dryer screen items #1-5 were mixed and blended by blending agitation with no heat.
2. The material was then blended and sprayed with a mixture of talc, triethyl citrate and Eudragit® 30% at 50 mL/kg of Eudragit® at 37° C. The temperature was not allowed to rise above 37° C. The blending was continued with dry air until the LOD was <1.20%.
3. The granulate was screened through a 093 D3 Fitzmill screen and then blended with dry air in the fluid bed dryer. The Carbopol® 974 was added and agitated for 3 min or until fully blended with the granulate.
4. Next, the magnesium stearate was added to the granulate and blended in a similar fashion until dry and free flowing.
5. The dried granulate was then removed and placed on a rotary press with oblong punches. The granulate was compressed into tablets weighing 950 mg and having a fracture force strength of 12-15 kg.

### Example 4

In another embodiment, predominately natural excipients were used to prolong the release of the HCA from the tablet matrix. In this formulation, polyvinyl acetate was used as the retardant pH sensitive releasing polymer. All other excipients were common USP ingredients. A formulation of the following composition (see Table 4) was prepared:

TABLE 4

| | | Amount | |
|---|---|---|---|
| Item # | Ingredient | (mg/Tablet) | Percent |
| 1 | HCA potassium/magnesium salt | 1,000 | 66.70 |
| 2 | Di-calcium phosphate | 174 | 11.60 |
| 3 | TPGS | 40 | 2.60 |
| 4 | Zein | 21 | 1.40 |
| 5 | Alginate (Satialgine) | 49.5 | 3.30 |
| 6 | Pectin | 60 | 4.00 |
| 7 | Glycerin | 100.5 | 6.70 |
| 8 | Polyvinyl acetate phthalate (PVAP) | 45 | 3.00 |
| 9 | Magnesium stearate | 10 | 0.70 |
| | TOTAL | 1,500 | 100 |

The method of preparation was as follows:
1. HCA and di-calcium phosphate were blended in a fluid bed dryer. When the HCA and di-calcium phosphate were blended, the TPGS was melted under heat until it was free-flowing and molten. The molten TPGS was then sprayed into the mixture of HCA and di-calcium phosphate. The TPGS-containing mixture was further blended in fluid bed dryer until a hard granulate formed.
2. Zein was dissolved in 250 mL of methanol alcohol (reagent grade) until the zein was well-dispersed.
3. The hardened mixture of step 1 was then granulated in a fluid bed with the fluidized zein and methanol.
4. While the zein and dry material were blended, pectin was prepared. Pectin was prepared by suspending the pectin into glycerin using a high shear mixer until the pectin was thoroughly blended and smooth in texture. The blended pectin was then slowly sprayed into the zein-coated HCA. The pectin-containing, zein-coated mixture was blended continuously until an even distribution of the components was achieved. The mixture was further blended until a distinct granulate formed. Blending was continued until the granulate was dry and well-formed.
5. For the last step in the granulation, screened alginate was added into the mixture. The blending was continued at 30° C. temperature for 15 min after the screened alginate was added. The alginate-containing granulate was blended until dry with a loss on drying (LOD)<1.5%.
6. The granulate was sized by passing the granulate material through a #120 Fitzmill screen. The sized granulate was then replaced into fluid bed dryer. PVAP was then sprayed onto the sized and dried granulate at room temperature while the granulate was agitated. The PVAP was prepared by dispersing it in 300 mL of purified water with 30 mL of NH₃OH. The entire lot of PVAP was sprayed onto the granulate and the material kept in the fluid bed dryer until the LOD was <1.2%.
7. The granulate was removed and passed through a 093 Fitzmill screen prior to blending it with magnesium stearate.
8. The screened granulate was then placed on a rotary press and compressed into oblong tablets weighing 1,500 mg and having a fracture force strength of 12±4 kg.

### Example 5

For the purposes of production, it was useful to have a standardized enteric starting material which has been stabilized for handling purposes and which could then be modified as to its release rate through the addition of ingredients, changes in handling and other techniques. Table 5 gives one formula that adds 5% solids from the coating to the starting HCA material. This means that a starting HCA salt yielding 65% HCA can be added to formulas as an enteric-coated granulate and calculated as 60% HCA. Kollicoat® IR (polyvinylalcohol-polyethyleneglycol graft-copolymer) is an instant-release coating useful to create an HCA granulate composition for further processing that does not immediately become gummy when subjected to moisture and other challenges. Eastacryl® from Eastman is a dispersion of CAP (cellulose acetate pthalate) used to provide a sustained-release coating.

TABLE 5
Enteric Coating using 2% Kollicoat IR and 3% Eastacryl

| Item # | Ingredient | Wt (Kg) | Percent |
|---|---|---|---|
| 1 | HCA potassium/magnesium salt (65% HCA) | 2.000 | 76.34 |
| 2 | Kollicoat IR | 0.040 | 1.53 |
| 3 | Water | 0.380 | 14.50 |
| 4 | Eastacryl (30% solids) | 0.2 | 7.63 |
| | (yielding dissolved CAP solids) | (0.06) | |
| | TOTAL | 2.62 | 100.00 |

The method of preparation was as follows:
1. Items #1-3 were weighed and blended in a fluid bed dryer with Kollicoat® IR as follows: Kollicoat IR was dissolved in 400 ml water. Roughly 15 ml of alcohol was added to aid in drying. Product then was spray dried onto the HCA salt in a fluid bed dryer with a temperature <50° C. until moisture content was approximately 2.5%.
2. The material produced in step 1 next was coated with Eastacryl® in a fluid bed dryer to give it enteric characteristics.
3. Coating technique information for the spray dryer for the Eastacryl was as follows:
   Spray rate: 9%
   Outlet: 25-33° C.
   Inlet: 45-55° C.
   Atomizer: 55 PSI
   CFM: 200-400

Dried to: 45° C. outlet temperature; inlet less than 60° C.
4. With the loss of added water, the resulting enteric granulate, referred to as HCActive™ (60%) Enteric Granulation, yields 60% HCA and was suitable for incorporation into specific formulations of the present invention.

### Example 6

The procedure in Example 5 yielded a relatively durable granulate. For some purposes, an adequate enteric powder can be produced utilizing magnesium stearate or similar compounds. Such a procedure requires less equipment and less time.

TABLE 6
Enteric Coating using 5% Magnesium Stearate

| Item # | Ingredient | Wt (Kg) | Percent |
|---|---|---|---|
| 1 | HCA potassium/magnesium salt (65% HCA) | 2.000 | 95.24 |
| 2 | Magnesium Stearate | 0.100 | 4.76 |
| | TOTAL | 2.10 | 100.00 |

The method of preparation was as follows:
1. Items #1 and 2 were weighed and blended thoroughly.
2. The material produced in step 1 next was heated to approximately 35° C. while blending continued. This step was continued long enough to melt the magnesium stearate and coat the HCA salt evenly.
3. Blending was maintained until the granulate had cooled to approximately room temperature.
4. The resulting granulate was screened through a 093 D3 Fitzmill screen to control the size of the particles. The HCA content of the resulting granulate was approximately 60%.

### Example 7

In another embodiment, the HCActive™ (60%) Enteric Granulation produced in Example 5 was used to create an extended-release enteric formulation that included TPGS. Additional delivery control came from the inclusion of Kollicoat® SR (polyvinylacetate dispersion stabilized with povidone and sodium laurylsulfate). Kollicoat® SR provided a sustained-release coating.

TABLE 7
Extended Release (with Enteric and TPGS)

| | | | Amount | | |
|---|---|---|---|---|---|
| Item # | Item Key | Ingredient | (mg/Tablet) | Wt (Kg) | Percent |
| 1 | Premix | HCActive ™ | 833.33 | 1.000 | 57.391 |
| | | (60%) Enteric | | | |
| | | Granulation | | | |
| 2 | | TPGS | 167 | 0.200 | 11.501 |
| 3 | | Kollicoat SR | 12.5 | 0.015 | 0.861 |
| 4 | | Silicon dioxide | 167 | 0.200 | 11.501 |
| | | (4% Aerosil) | | | |
| 5 | | Starch | 15 | 0.018 | 1.033 |
| 6 | | Mag Stearate | 7 | 0.0234 | 0.502 |
| 7 | | Di-calcium | 250 | 0.300 | 17.219 |
| | | Phosphate | | | |
| | | TOTAL | 1451.83 | 1.7564 | 100.008 |

The method of preparation was as follows:
1. The Premix HCActive™ (60%) Enteric Granulation was produced as indicated in Example 5.
2. The molten TPGS was mixed with the Aerosil and then the product was mixed with the Premix before being added to the other ingredients and blended to achieve uniformity.
3. The resulting granulate was screened through a 093 D3 Fitzmill screen to control the size of the particles.
4. The powder was then removed and placed on a rotary press with oblong punches. It formed tablets readily. The granulate was compressed into tablets weighing approximately 1450 mg and having a fracture force strength of 12-15 kg.

There was little or no breakdown of these extended-release enteric formulation-containing tablets upon exposure with stirring to simulate gastric fluid without pepsin (HCl solution, pH 1.2) for 60 min. These extended-release enteric formulation-containing tablets dissolved completely in approximately 55 min when agitated in monophosphate buffer, pH 6.8 (as described previously in Example 1).

### Example 8

In another embodiment, the HCActive™ (60%) Enteric Granulation produced in Example 5 was used to create an extended-release enteric formulation that included TPGS. Unlike Example 6, in this example the TPGS was not first mixed with Aerosil, but rather liquefied and added to the total powder as described below.

TABLE 8
Extended Release (with Enteric and TPGS)

| | Item | | Amount | Wt | |
|---|---|---|---|---|---|
| Item # | Key | Ingredient | (mg/Tablet) | (Kg) | Percent |
| 1 | Premix | HCActive ™ | 833.33 | 1.000 | 57.391 |
| | | (60%) Enteric | | | |
| | | Granulation | | | |
| 2 | | TPGS | 167 | 0.200 | 11.501 |
| 3 | | Kollicoat SR | 12.5 | 0.015 | 0.861 |
| 4 | | Microcrystalline | 167 | 0.200 | 11.501 |
| | | Cellulose | | | |
| 5 | | Starch | 15 | 0.018 | 1.033 |
| 6 | | Magnesium | 7 | 0.0234 | 0.502 |
| | | Stearate | | | |
| 7 | | Di-calcium | 250 | 0.300 | 17.219 |
| | | Phosphate | | | |
| | | TOTAL | 1451.83 | 1.7564 | 100.008 |

The method of preparation was as follows:
1. The Premix HCActive™ (60%) Enteric Granulation was produced as indicated in Example 5.
2. Molten TPGS was added to the Premix while mechanical blending was taking place. The resultant mixture was an oily powder, improved for handling by the addition of microcrystalline cellulose, followed by the addition and blending of items #1, 3, 5, 6 into the whole.
3. The resulting granulate was screened through a 093 D3 Fitzmill screen to control the size of the particles.
4. The powder was then removed and placed on a rotary press with oblong punches. The granulate was compressed into tablets weighing approximately 1450 mg. However, the tablets were brittle and of uneven density.

These extended-release enteric formulation-containing tablets dissolved completely in approximately 55 min when agitated in monophosphate buffer, pH 6.8 (as described previously in Example 1).

### Example 9

In another embodiment, the HCActive™ (60%) Enteric Granulation produced in Example 5 was used to create an extended-release enteric formulation that included TPGS. Unlike Example 6, in this example the TPGS was first mixed with a smaller amount of Aerosil and then refrigerated overnight to improve handling.

TABLE 9
Extended Release (Enteric with TPGS and Aerosil)

| | Item | | Amount | | |
|---|---|---|---|---|---|
| Item # | Key | Ingredient | (mg/Tablet) | Wt (Kg) | Percent |
| 1 | Premix | HCActive ™ | 833.33 | 1.000 | 0.57339 |
| | | (60%) Enteric | | | |
| | | Granulation | | | |
| 2 | | TPGS | 167 | 0.200 | 0.11491 |
| 3 | | Aerosil | 61 | 0.073 | 0.04197 |
| 4 | | Magnesium | 7 | 0.0086 | 0.00482 |
| | | Stearate | | | |
| 5 | | Di-calcium | 385 | 0.462 | 0.26491 |
| | | Phosphate | | | |
| | | TOTAL | 1453.33 | 1.7436 | 1.00000 |

The method of preparation was as follows:
1. The Premix HCActive™ (60%) Enteric Granulation was produced as indicated in Example 5.
2. The TPGS was heated on a hot plate in stainless steel container and then added to the Aerosil and mixed with a Kitchen Aide blender to form a solid mass, then refrigerated overnight.
2. The next day, TPGS/Aerosil block was broken up and reduced to granulate, then this granulate was blended into the other ingredients.
3. The resulting material was screened through a 093 D3 Fitzmill screen to control the size of the particles.
4. The powder was then removed and placed on a rotary press with oblong punches. The granulate was compressed into tablets weighing approximately 1450 mg and having a fracture force strength of 12-15 kg.

These extended-release enteric formulation-containing tablets dissolved completely in approximately 45 min when agitated in monophosphate buffer, pH 6.8 (as described previously in Example 1).

### Example 10

In another embodiment, the HCActive™ (60%) Enteric Granulation produced in Example 5 was used to create an extended release enteric formulation that included Lubritab® in place of TPGS. Lubritab® could be mixed into the formulation as a dry powder and did not require the extensive pretreatment that TPGS needed.

TABLE 10
Extended Release (Enteric with Lubritab)

| | Item | | Amount | | |
|---|---|---|---|---|---|
| Item # | Key | Ingredient | (mg/Tablet) | Wt (Kg) | Percent |
| 1 | Premix | HCA (60%) | 833.33 | 1.000 | 55.543 |
| | | Enteric | | | |
| | | Granulation | | | |
| 2 | | Di-calcium | 332 | 0.376 | 22.128 |
| | | Phosphate | | | |
| 3 | | Lubritab | 228 | 0.258 | 15.197 |
| 4 | | Kollicoat SR | 100 | 0.113 | 6.665 |
| 5 | | Magnesium | 7 | 0.0082 | 0.466 |
| | | Stearate | | | |
| | | TOTAL | 1500.33 | 1.7552 | 99.999 |

The method of preparation was as follows:
1. The Premix HCActive™ (60%) Enteric Granulation was produced as indicated in Example 5.
2. All ingredients were blended together.
3. The resulting material was screened through a 093 D3 Fitzmill screen to control the size of the particles.
4. The powder was then removed and placed on a rotary press with oblong punches. The granulate was compressed into tablets weighing approximately 1500 mg and having a fracture force strength of 12-15 kg.

These extended-release enteric formulation-containing tablets took more than 12 h to dissolved completely when agitated in monophosphate buffer, pH 6.8 (as described previously in Example 1). The wicking action of Aerosil, microcrystalline cellulose or some similar component is useful to augment this enteric formulation in order to decrease the dissolution time. Using the formulations of the invention, HCA release rates could be controlled for periods of at least about 30 min to more than about 12 h at pH 6.8. Lubritab® was a useful substitute for TPGS in the HCA-containing compounds of the present invention.

### Example 11

### Comparative Analysis of the Chloride Content and Total Halogen Content of HCA-Containing Compound of the Present Invention and Another Commercial HCA-Containing Preparation

The chloride content of select HCA-containing preparations was determined by elemental and ion chromatographic analysis by Galbraith Laboratories, Inc. (Knoxville, Tenn.) as summarized in Tables 11 and 12 below. As shown in Table 11, the chloride content of an HCA-containing compound of the present invention (RH1-1) was at least 6-fold lower than the chloride content of a commercial HCA-containing preparation (SCM-1) according to ion exchange chromatography employing standard techniques satisfying Environmental Protection Agency (EPA) methods/EPA 300.0.

TABLE 11
Chloride Content

Weight Percent

| Sample ID | Test Sample 1 | Test Sample 2 |
|---|---|---|
| RH1-1 | 0.427 | 0.424 |
| SCM-1 | 2.71 | 2.65 |

Chloride content is tightly controlled in many countries for health reasons. The HCA-containing compound was produced using the methods previously described in U.S. Pat. Nos. 5,656,314 and 5,536,516 and then further processed as follows. Briefly, a solution of HCA-containing compound was passed over a small volume of strong anion exchange column where preferentially chlorides are bound along with HCA. Minimum amount of HCA is lost but the chlorides are reduced considerably so as to achieve chloride levels of less than about 0.6%. Afterward, this solution is treated with charcoal and reacted with magnesium and potassium according to our art, to get a Mg—K HCA which is subsequently spray-dried to derive less hygroscopic free-flowing powder. In one embodiment of the present invention, elemental magnesium and elemental potassium are present in the HCA-containing compound in a ratio of between about 1:10 to about 1:3.

Halogen refers to those elements in the seventeenth column of the periodic table: fluorine (F), chlorine (Cl), bromine (Br), iodine (I), and astatine (At). Halogenated refers to a chemical compound or mixture that contains halogen atoms. In a covalent molecule, the halide atom has a strong, directional chemical bond to another atom. If this other atom is a carbon atom the material is a halogenated organic molecule, e.g., carbon tetrachloride, methylene chloride (dichloromethane), trichloroethylene, polyvinyl chloride (PVC). Halogenated organic molecules are a very important class of chemicals that are used to produce a wide variety of other chemicals and consumer products. When a covalent halide dissolves, the halogen atom remains firmly attached to whatever it was bonded to and is not an electrolyte. Chlorinated organic molecules are often health hazards and some are even known human carcinogens. Generally, the more chlorine atoms an organic molecule has, the more likely it is to be carcinogenic. Accordingly, the total halogens as chloride content of select HCA-containing preparations was determined by Galbraith Laboratories, Inc. (Knoxville, Tenn.) as summarized in Table 12 below.

Total elemental chlorine was determined using the Environmental Protection Agency (EPA) method/EPA 330.5 (yielding total residual chlorine). Elemental analysis is superior to ion analysis in cases in which chlorine is molecularly bound such as to not be readily released through oxidation or other techniques and in certain other instances. The findings for both samples with elemental analysis were slightly higher than those with ion determination. As shown in Table 12, the total halogens as chloride content of an HCA-containing compound of the present invention (RH1-1) was at least 5-fold lower than the total halogens as chloride content of a commercial HCA-containing preparation (SCM-1).

TABLE 12
Total Halogens as Chloride Content

Weight Percent

| Sample ID | Test Sample 1 | Test Sample 2 |
|---|---|---|
| RH1-1 | 0.603 | 0.599 |
| SCM-1 | 3.02 | 3.05 |

### Example 12

### Testing the HCA-Containing Compounds in a Rat Model

An OM rat model is useful to test the biological properties of the HCA-containing compounds of the invention. Briefly, male OM rats aged 10 weeks are fed a diet in which 30% of the calories are obtained from fat under standard conditions. Groups of 5-10 rats are intubated twice daily with HCA-containing test compound (e.g., 0.01 mmoles/kg body weight to 1 mole/kg body weight) or placebo for 60 days. Blood is withdrawn from the tail vein one or more times daily. The pharmacokinetics of HCA-containing compounds, including absorption, is determined by measuring the HCA level in the blood of subjects administered the HCA-containing compound using gas chromatography/mass spectroscopy technique (Loe et al., Anal Biochem. 2001, 1; 292(1): 148-54) and as further detailed by Loe of al., (FASEB Journal, 2001, 15 4:632, Abs. 501.1). Body weight of the test subjects as well as, blood levels of lipids, hormones and metabolic regulators are measured, e.g., but not limited to, LDL and HDL, glucocorticoids, leptin, insulin, and corticosterone level (see generally, U.S. Pat. No. 6,482,858, issued Nov. 19, 2002). At the end of the 60 day experimental period, the animals are sacrificed. Experimental parameters such as body weight of the test subjects as well as, blood levels of lipids, hormones and metabolic regulators are measured, e.g., but not limited to, LDL and HDL, glucocorticoids, leptin, insulin, and corticosterone level in test subjects receiving HCA-containing compound is compared with these experimental parameters in subjects receiving placebo by statistical analysis using the Students t-test (one- or two-tailed P-values) or ANOVA. A P-value of less than or equal to about 0.05 is considered statistically significant. A statistically significant alteration, e.g., increase or decrease, in an experimental parameter of test subjects receiving HCA-containing compound compared to subjects receiving placebo indicates that the HCA-containing compound is a drug capable of the prevention or treatment of diseases or conditions characterized by alterations in such parameters.

### EQUIVALENTS

From the foregoing detailed description of the invention, it should be apparent that unique HCA-containing compounds and methods of the same have been described resulting in improved HCA-containing formulations suitable for therapeutic use. Although particular embodiments have been disclosed herein in detail, this has been done by way of example for purposes of illustration only, and is not intended to be limiting with respect to the scope of the appended claims which follow. In particular, it is contemplated by the inventor that substitutions, alterations, and modifications may be made to the invention without departing from the spirit and scope of the invention as defined by the claims. For instance, the choice of HCA salt, encapsulating agent or the choice of appropriate patient therapy based on these is believed to be matter of routine for a person of ordinary skill in the art with knowledge of the embodiments described herein.

1. A (−)-hydroxycitrate-containing composition, comprising:
(a) (−)-hydroxycitrate;
(b) one or more absorption-enhancer/controlled-release agents; and
(c) one or more rate-controlling excipients.

2. The (−)-hydroxycitrate-containing composition of claim 1, wherein the (−)-hydroxycitrate is selected from a group consisting of: (−)-hydroxycitrate free acid; (−)-hydroxycitrate salts; and (−)-hydroxycitrate derivatives, or any combination thereof.

3. The (−)-hydroxycitrate-containing composition of claim 1, wherein the (−)-hydroxycitrate is present from about 1.0% to about 80% of the total weight of the (−)-hydroxycitrate-containing composition.

4-5. (canceled)

6. The (−)-hydroxycitrate-containing composition of claim 1, wherein the one or more absorption-enhancer/controlled-release agents are selected from the group consisting of: d-alpha-tocopheryl polyethylene glycol succinate (TPGS); Lubritab®; volcanic oils; high viscosity grades of conjugated polyethylene glycol; ethylcellulose, carboxymethylcellulose, cellulose propionate; cellulose acetate propionate; cellulose acetate butyrate; cellulose acetate phthalate (CAP); cellulose triacetate; hydroxypropyl-methylcellulose phthalate; polymethyl methacrylate; polyethyl methacrylate; polybutyl methacrylate; polyisobutyl methacrylate; polyhexyl methacrylate; polyisodecyl methacrylate; polylauryl methacrylate; polyphenyl methacrylate; polymethyl acrylate; polyisopropyl acrylate; polyisobutyl acrylate; polyoctadecyl acrylate; polyethylene; polyethylene low density; polyethylene high density; polypropylene; polyethylene oxide; polyethylene terephthalate; polyvinyl isobutyl ether; polyvinyl acetate; polyvinyl acetate phthalate; polyvinyl chloride; polyurethane; other copolymers of acrylic and methacrylic and esters; waxes; shellac; zein; hydrogenated vegetable oils; polyvinyl alcohol; polyvinylpyrrolidone; methyl cellulose; hydroxypropyl cellulose; hydroxpropylmethyl cellulose or polyethylene glycol; or a mixture thereof.

7. The (−)-hydroxycitrate-containing composition of claim 1, wherein the one or more absorption-enhancer/controlled-release agents are present from about 1.0% to about 50% of the total weight of the (−)-hydroxycitrate-containing composition.

8-9. (canceled)

10. The (−)-hydroxycitrate-containing composition of claim 1, wherein the one or more rate-controlling excipients are selected from the group consisting of: Eastacryl; Kollicoat® IR (polyvinylalcohol-polyethyleneglycol graft-copolymer); cellulose acetate phthalate; Kollicoat® SR; ethyl cellulose; Eudragit® (family of acrylate and methacrylate-based coatings); zein (vegetable protein); acrylic polymers; polyvinyl acetate phthalate; hydroxymethylpropylmethyl cellulose phthalate; cellulose acetate trimalleate; acrylic polymer plasticizers; polymers of polylactic acid; polymers of glycolic acid, and mixtures thereof; Primogel; Pruv™ (stearyl fumarate sodium); citrate esters; triethyl citrate; propylene glycol; and dibutyl sebacate.

11. The (−)-hydroxycitrate-containing composition of claim 1, wherein the one or more rate-controlling excipients are present from about 0.0001% to about 60% of the total weight of the (−)-hydroxycitrate-containing composition.

12-19. (canceled)

20. The (−)-hydroxycitrate-containing composition of claim 1 further comprising one or more lubricants.

21. The (−)-hydroxycitrate-containing composition of claim 20, wherein the one or more lubricants are selected from a group consisting of: magnesium stearate, calcium stearate; sodium stearate, glycerol monostearate; stearic acid; Lubritab®; hydrogenated vegetable oils; waxes; talc; boric acid; sodium benzoate; sodium acetate; sodium chloride; DL-leucine; sodium oleate; sodium lauryl sulfate; magnesium lauryl sulfate and polyethylene glycols and kaolin.

22. The (−)-hydroxycitrate-containing composition of claim 20, wherein the one or more lubricants are present from about 0.0001% to about 10% of the total weight of the of the (−)-hydroxycitrate-containing composition.

23-30. (canceled)

31. The (−)-hydroxycitrate-containing composition of claim 1 further comprising one or more bulking agents/binders.

32. The (−)-hydroxycitrate-containing composition of claim 31, wherein the one or more bulking agents/binders are selected from a group consisting of: starch paste; acacia; sucrose; poly vinyl pyrrolidone (PVP); hydroxy proplyl methyl cellulose (HPMC); methyl cellulose; gelatin; potato starch; micro crystalline cellulose (MCC); pregelatinized starch (PGS); Primogel (Sodium starch glycolate, USP/NF, Ph. Eur.); Primellose (Crosscarmelose sodium, USP/NF, ph. Eur.); di-calcium phosphate and tri-calcium phosphate.

33. The (−)-hydroxycitrate-containing composition of claim 31, wherein the one or more bulking agents/binders are present from about 0.01% to about 30% of the total weight of the of the (−)-hydroxycitrate-containing composition.

34-41. (canceled)

42. The (−)-hydroxycitrate-containing composition of claim 1 further comprising one or more lubricants and one or more bulking agents/binders.

43. The (−)-hydroxycitrate-containing composition of claim 42, wherein the one or more lubricants are selected from a group consisting of: magnesium steareate; Lubritab®; talc; glycerol monostearate; and kaolin.

44. The (−)-hydroxycitrate-containing composition of claim 42, wherein the one or more lubricants are present from about 0.0001% to about 10% of the total weight of the of the (−)-hydroxycitrate-containing composition.

45-46. (canceled)

47. The (−)-hydroxycitrate-containing composition of claim 42, wherein the one or more bulking agents/binders are selected from a group consisting of: starch paste; acacia; sucrose; poly vinyl pyrrolidone (PVP); hydroxy proplyl methyl cellulose (HPMC); methyl cellulose; gelatin; potato starch; micro crystalline cellulose (MCC); pregelatinized starch (PGS); Primogel (Sodium starch glycolate, USP/NF, Ph. Eur.); Primellose (Crosscarmelose sodium, USP/NF, ph. Eur.); di-calcium phosphate and tri-calcium phosphate.

48. The (−)-hydroxycitrate-containing composition of claim 42, wherein the one or more bulking agents/binders are present from about 0.01% to about 30% of the total weight of the of the (−)-hydroxycitrate-containing composition.

49-50. (canceled)

51. The (−)-hydroxycitrate-containing composition of claim 42, wherein the chloride concentration is less than about 2.5% of the total weight of the (−)-hydroxycitrate-containing composition.

52-53. (canceled)

54. The (−)-hydroxycitrate-containing composition of claim 42, wherein the total halogen content as chloride is less than about 2.9% of the total weight of the (−)-hydroxycitrate-containing composition.

55-188. (canceled)
